(19) United States
(12) Patent Application Publication
(10) Pub. No.: US 2026/0260768 A1
(43) Pub. Date: Sep. 3, 2026

(54) INFORMATION PROCESSING APPARATUS, INFORMATION PROCESSING METHOD, AND NON-TRANSITORY COMPUTER-READABLE MEDIUM

(71) Applicants: NEC Corporation, Tokyo (JP); SOCIAL WELFARE ORGANIZATION SAISEIKAI IMPERIAL GIFT FOUNDATION, INC. SAISEIKAI KUMAMOTO HOSPITAL, Kumamoto-shi (JP)

(72) Inventors: Konosuke TEMMEI, Tokyo (JP); Shuhei Noyori, Tokyo (JP); Kei shibuya, Tokyo (JP); Keiichirou Okamoto, Tokyo (JP); Miekio Mano, Tokyo (JP); Sumihiro Hirai, Kumamoto (JP); Hideyuki Tanaka, Kumamoto (JP); Shinji Takamune, Kumamoto (JP); Rui Sugeta, Kumamoto (JP)

(73) Assignees: NEC Corporation, Tokyo (JP); SOCIAL WELFARE ORGANIZATION SAISEIKAI IMPERIAL GIFT FOUNDATION, INC. SAISEIKAI KUMAMOTO HOSPITAL, Tokyo (JP)

(21) Appl. No.: 19/553,701

(22) Filed: Mar. 2, 2026

(30) Foreign Application Priority Data
Mar. 3, 2025 (JP) 2025-032566

(51) Int. Cl. G16H 50/70; G16H 10/60

(52) U.S. Cl. CPC G16H 50/70; G16H 10/60

(57) ABSTRACT

To more appropriately support doctors and the like based on similar cases. Provided is an information processing apparatus including an acquisition unit that acquires clinical information of a plurality of patients, and a control unit that extracts clinical information of another patient whose similarity to the clinical information of a specific patient is equal to or greater than a threshold, and outputs information generated from the clinical information of the other patient based on a sentence instructed by a user.

### INCORPORATION BY REFERENCE

This application is based upon and claims the benefit of priority from Japanese patent application No. 2025-032566, filed on Mar. 3, 2025, the disclosure of which is incorporated herein in its entirety by reference.

### TECHNICAL FIELD

The present disclosure relates to an information processing apparatus, an information processing method, and a program.

### BACKGROUND ART

Techniques for searching for similar cases are known (for example, JP 2008-217362 A).

### SUMMARY

However, in the technique described in JP 2008-217362 A, for example, there is room for improvement in supporting doctors and the like based on similar cases.

In view of the above-described problems, an example object of the present disclosure is to provide a technique capable of more appropriately supporting doctors and the like based on similar cases.

According to a first example aspect of the present disclosure, there is provided an information processing apparatus including an acquisition unit that acquires clinical information of a plurality of patients, and a control unit that extracts clinical information of another patient whose similarity to the clinical information of a specific patient is equal to or greater than a threshold, and outputs information generated from the clinical information of the other patient based on a sentence instructed by a user.

According to a second example aspect of the present disclosure, there is provided an information processing method including acquiring clinical information of a plurality of patients, extracting clinical information of another patient whose similarity to the clinical information of the specific patient is equal to or greater than a threshold, and outputting information generated from the clinical information of the other patient based on a sentence instructed by the user.

According to a third example aspect of the present disclosure, there is provided a program including acquiring clinical information of a plurality of patients, extracting clinical information of another patient whose similarity to the clinical information of the specific patient is equal to or greater than a threshold, and outputting information generated from the clinical information of the other patient based on a sentence instructed by the user.

According to one aspect, it is possible to more appropriately support doctors and the like based on similar cases.

### BRIEF DESCRIPTION OF DRAWINGS

The above and other aspects, features and advantages of the present disclosure will become more apparent from the following description of certain exemplary embodiments, taken in conjunction with the accompanying drawings, in which:

FIG. 1 is a diagram illustrating an example of a configuration of an information processing apparatus according to the present disclosure;

FIG. 2 is a diagram illustrating an exemplary hardware configuration of the information processing apparatus according to the present disclosure;

FIG. 3 is a flowchart illustrating an example of a process of the information processing apparatus according to the present disclosure;

FIG. 4 is a diagram illustrating an example of information stored in a clinical information database (DB) according to the present disclosure;

FIG. 5 is a diagram illustrating an example of a setting screen of a feature item according to the present disclosure;

FIG. 6 is a diagram illustrating an example of a display screen according to the present disclosure; and

FIG. 7 is a diagram illustrating an example of a display screen according to the present disclosure.

### EXAMPLE EMBODIMENT

The principles of the present disclosure will be described with reference to several exemplary example embodiments. It is to be understood that the example embodiments have been described for purposes of exemplification only and will aid those of ordinary skill in the art in understanding and carrying out the present disclosure, without suggesting any limitations on the scope of the present disclosure. The disclosure described in the present description is implemented in various methods other than those described below.

In the following description and claims, unless defined otherwise, all technical and scientific terms used in the present specification have the same meaning as commonly understood by those skilled in the art of the technical field to which the present disclosure belongs.

Hereinafter, example embodiments of the present disclosure will be described with reference to the drawings. Each of the drawings is merely an example to illustrate one or more example embodiments. Each of the drawings is not associated with only one specific example embodiment, but may be associated with one or more other example embodiments. As those of ordinary skill in the art will appreciate, various features or steps described with reference to any one of the drawings may be combined with features or steps illustrated in one or more other drawings, for example, to create an example embodiment that is not explicitly illustrated nor described. All of the features or steps illustrated in any one of the drawings for describing illustrative example embodiments are not necessarily mandatory, and some features or steps may be omitted. The order of the steps described in any of the drawings may be changed as appropriate.

### Configuration

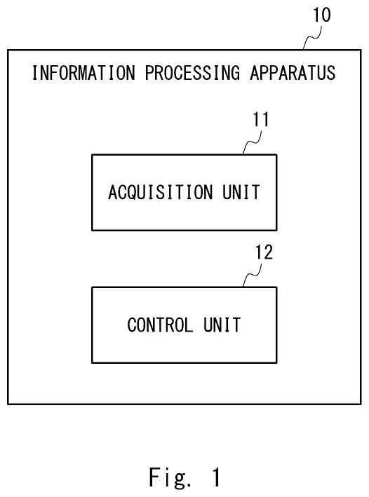

A configuration of an information processing apparatus 10 according to an example embodiment will be described with reference to FIG. 1. FIG. 1 is a diagram illustrating an example of the configuration of the information processing apparatus 10 according to the example embodiment. The information processing apparatus 10 includes an acquisition unit 11 and a control unit 12. These units may be achieved by cooperation of one or more programs installed in the information processing apparatus 10 and hardware such as a processor and a memory of the information processing apparatus 10.

The acquisition unit 11 acquires clinical information of a plurality of patients. The control unit 12 extracts clinical information of another patient whose similarity to the clinical information of the specific patient is equal to or greater than a threshold, and outputs information generated from the clinical information of the other patient based on a sentence instructed by the user. This makes it possible to more appropriately support doctors and the like based on similar cases.

### Hardware Configuration

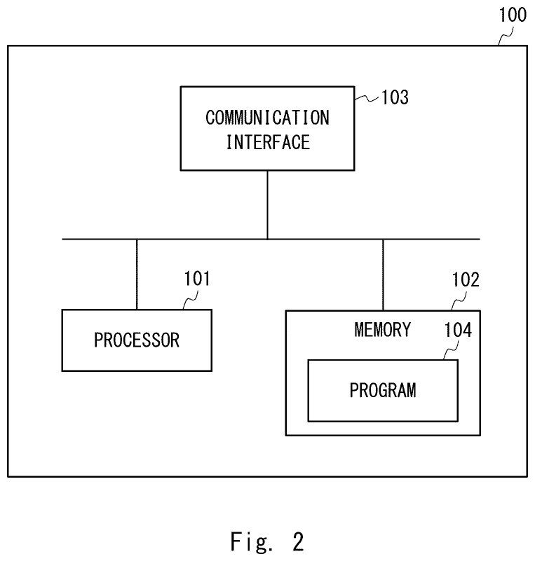

FIG. 2 is a diagram illustrating a hardware configuration example of the information processing apparatus 10 according to the example embodiment. In the example in FIG. 2, the information processing apparatus 10 (computer 100) includes a processor 101, a memory 102, and a communication interface 103. These units may be connected by a bus or the like. The memory 102 stores at least a part of a program 104. The communication interface 103 includes an interface necessary for communication with other network elements.

In a case where the program 104 is executed by the cooperation of the processor 101, the memory 102, and the like, at least a part of processing according to the example embodiment of the present disclosure is performed by the computer 100. The memory 102 may be of any type. The memory 102 may be a non-transitory computer-readable storage medium, as a non-limiting example. The memory 102 may also be implemented using any appropriate data storage technique such as a semiconductor-based memory device, magnetic memory device and system, optical memory device and system, a fixed memory, and a removable memory. Although only one memory 102 is illustrated in the computer 100, there may be several physically different memory modules in the computer 100. The processor 101 may be of any type. The processor 101 may include one or more of a general-purpose computer, a dedicated computer, a microprocessor, a digital signal processor (DSP), and a processor based on a multi-core processor architecture as a non-limiting example. The computer 100 may have a plurality of processors such as an application specific integrated circuit chip that is temporally dependent on a clock that synchronizes a main processor.

Example embodiments of the present disclosure may be implemented in hardware or dedicated circuitry, software, logic, or any combination thereof. Some aspects may be implemented in hardware, while other aspects may be implemented in firmware or software that may be executed by a controller, a microprocessor, or other computing devices.

The present disclosure also provides at least one computer program product tangibly stored on a non-transitory computer-readable storage medium. The computer program product includes computer-executable instructions, such as instructions included in a program module, and is executed on a related device on a real or virtual processor to execute the processes or methods of the present disclosure. The program module includes routines, programs, libraries, objects, classes, components, data structures, and the like that, for example, execute specific tasks or implement specific abstract data types. Functions of the program module may be combined or divided between program modules as desired in various example embodiments. A machine-executable instruction of the program module can be executed in a local or distributed device. In the distributed device, the program modules can be located on both local and remote storage media.

Program codes for executing the methods of the present disclosure may be written in any combination of one or more programming languages. These program codes are provided to a processor or controller of a general-purpose computer, a dedicated computer, or other programmable data processing devices. In a case where the program codes are executed by the processor or controller, the functions/operations in the flowcharts and/or the implemented block diagrams are executed. The program code is executed entirely on a machine, partly on the machine as a stand-alone software package, partly on the machine and partly on a remote machine, or entirely on the remote machine or a server.

The program includes instructions (or software codes) for causing the computer to perform one or more functions described in the example embodiment in a case of being read by the computer. The programs may be stored in a non-transitory computer-readable medium or a tangible storage medium. As an example and not by way of limitation, the computer-readable medium or the tangible storage medium includes a random-access memory (RAM), a read-only memory (ROM), a flash memory, a solid-state drive (SSD) or any other memory technology, a CD-ROM, a digital versatile disc (DVD), a Blu-ray (registered trademark) disc or any other optical disc storage, and a magnetic cassette, a magnetic tape, a magnetic disk storage, or any other magnetic storage device. The program may be transmitted through a transitory computer-readable medium or a communication medium. As an example and not by way of limitation, the transitory computer-readable medium or the communication medium includes propagated signals in electrical, optical, acoustic, or any other form.

### Process

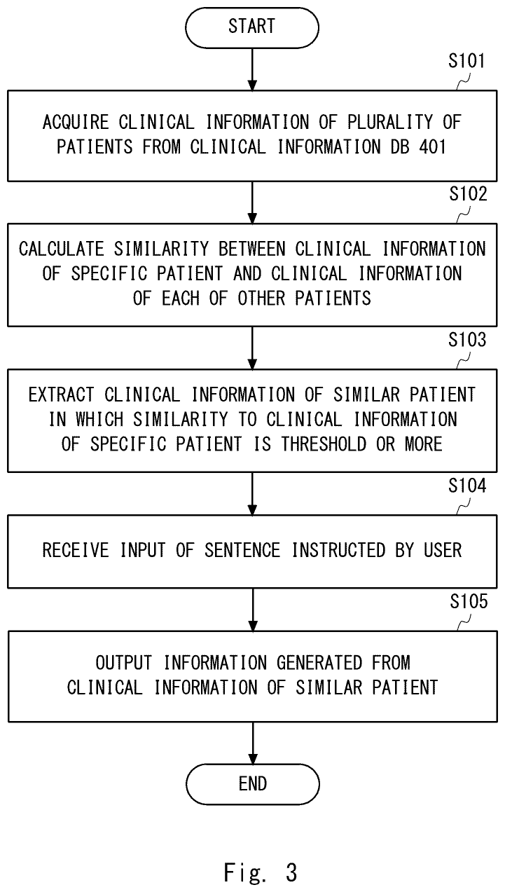

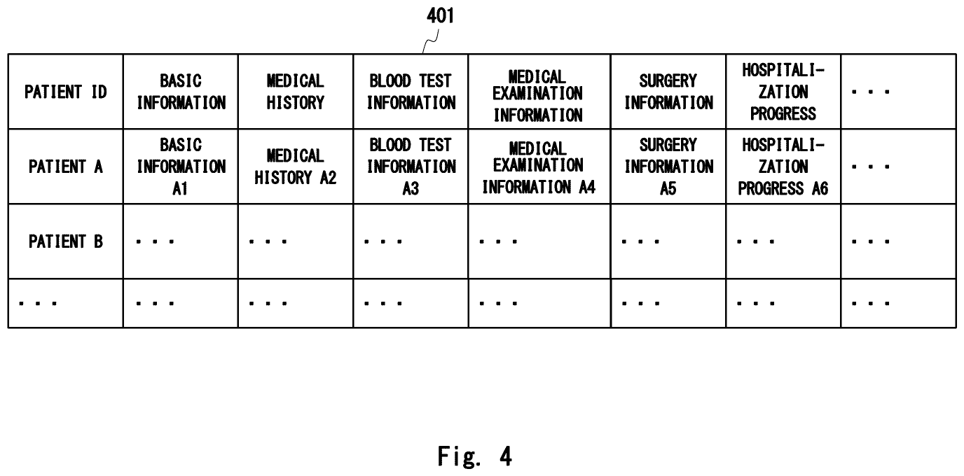

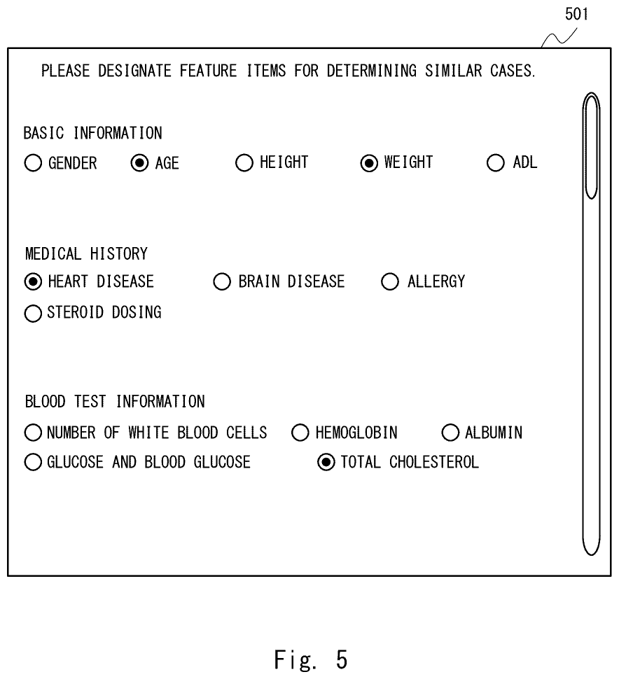

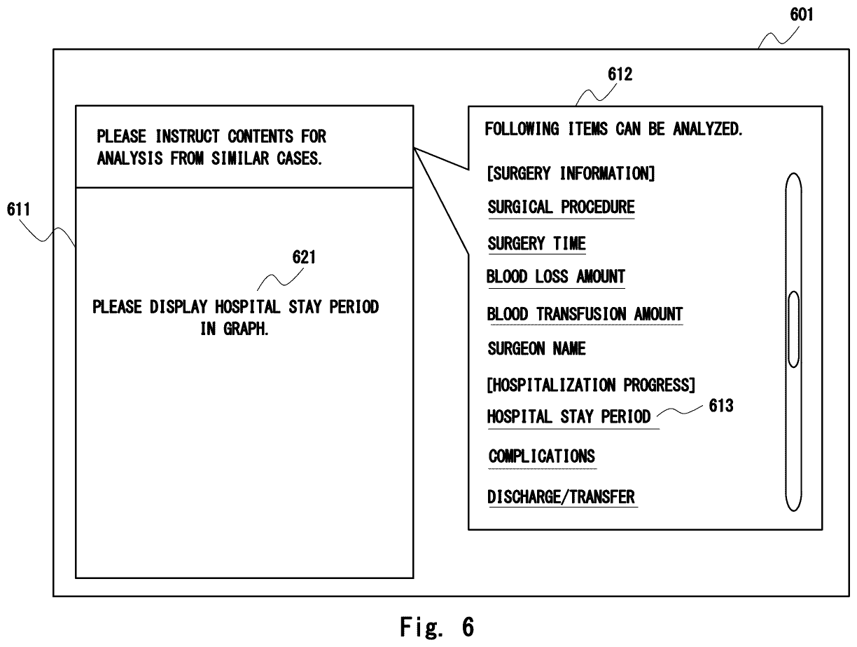

Next, an example of a process of the information processing apparatus 10 according to the example embodiment will be described with reference to FIG. 3 to 6. FIG. 3 is a flowchart illustrating an example of the process of the information processing apparatus 10 according to the example embodiment. FIG. 4 is a diagram illustrating an example of information stored in a clinical information database (DB) 401 according to the example embodiment. FIG. 5 is a diagram illustrating an example of a feature item setting screen according to the example embodiment. FIGS. 5 and 6 are diagrams illustrating an example of a display screen according to the example embodiment. The process of FIG. 3 may be executed in response to an operation by a user (for example, a doctor or the like), for example.

In step S101, the acquisition unit 11 acquires clinical information of a plurality of patients from the clinical information DB 401. The data recorded in the clinical information DB 401 may be generated based on, for example, information acquired from a data warehouse of medical information. The clinical information DB 401 may be recorded in a storage device inside the information processing apparatus 10 or may be recorded in a storage device outside the information processing apparatus 10.

In the example of FIG. 4, basic information, medical history, blood test information, medical examination information, surgery information, and information indicating the hospitalization progress are recorded in the clinical information DB 401 in association with the patient ID.

The patient ID is identification information of a patient. The basic information may include, for example, information of items such as gender, age, height, weight, and activities of daily living (ADL). The medical history may include, for example, information of a medical history of items such as heart disease, brain disease, allergy, and steroid medication.

The blood test information may include, for example, information on items such as the number of white blood cells, hemoglobin, albumin, glucose, blood sugar, and total cholesterol. The medical examination information may include, for example, information on items such as a main disease name, TNM (T: tumor, N: lymph nodes, M: metastasis) classification that is an index for evaluating the degree of progression of cancer, lung function, electrocardiogram findings, and chest X-ray findings. The surgery information may include, for example, information of items such as a surgical procedure, a surgery time, a blood loss amount, a blood transfusion amount, and a surgeon's name. The hospitalization progress may include, for example, information on items such as a hospital stay period, complications, and discharge/transfer.

Subsequently, the control unit 12 calculates similarity between the clinical information of the specific patient and the clinical information of each of the other patients (step S102). The specific patient may be, for example, a special person designated by the user. The control unit 12 may vectorize values of one or more items included in the clinical information of each patient and calculate similarity between the clinical information of the specific patient and the clinical information of other patients in a vector space. In this case, the similarity may be, for example, cosine similarity. The similarity may be calculated (inferred) using, for example, supervised learning or unsupervised learning.

The control unit 12 may calculate the similarity between the clinical information of the specific patient and the clinical information of each of the other patients based on one or more items (feature items) instructed by the user among the items included in the clinical information. As a result, for example, the user can designate a viewpoint (item) for determining as a similar case. Therefore, even in a case where the value of the specific item of the specific patient is a remarkable (feature) value, or the like, similar cases can be more appropriately detected. In the example of FIG. 5, on a setting screen 501, items of age, weight, heart disease, and total cholesterol are designated (selected) by the user as feature items used for determining similarity.

The control unit 12 may receive a setting of a feature item used for determination of similarity for each of at least one of a clinical department and a disease name. Then, the control unit 12 may calculate similarity between the clinical information of the specific patient and the clinical information of each of the other patients based on one or more set feature items among the items included in the clinical information. As a result, for example, even in a case where items to be used for similarity determination are different depending on clinical departments or the like, similar cases can be more appropriately detected.

Subsequently, the control unit 12 extracts clinical information of another patient (hereinafter, the patient is also appropriately referred to as a “similar patient”) whose similarity to the clinical information of the specific patient is equal to or greater than a threshold (step S103). Subsequently, the control unit 12 receives an input of a sentence (text) instructed by the user (step S104).

In a case where a sentence is instructed by the user, the control unit 12 may display information indicating an item other than the item used for similarity determination among a plurality of items included in the clinical information of the similar patient. As a result, for example, it can support the user to analyze the value of an item not used for determination of a similar case among the items of the specific patient.

In this case, in a case where an input field (text box) 611 of the instruction sentence illustrated in FIG. 6 is designated by a mouse operation or the like, the control unit 12 displays a display region 612 in association with the input field 611. In the display region 612, a list of items other than the items used for similarity determination is displayed.

The control unit 12 may determine and display a candidate of the instruction sentence relevant to the item designated by the user among the plurality of items included in the clinical information of the similar patient. As a result, for example, it is possible to support the input of the instruction sentence by the user. The candidate sentence of the instruction sentence relevant to the item may be set in the information processing apparatus 10 by the operator or the like, or may be generated using artificial intelligence (AI) such as a large language model.

In this case, in a case where the item 613 of the “hospital stay period” is designated by the user by a click operation or the like in the display region 612 illustrated in FIG. 6, the control unit 12 may display the sentence of the candidate 621 of the instruction sentence relevant to the item of the “hospital stay period” in the input field 611. The control unit 12 may enable the user to appropriately correct the sentence in the input field 611.

The control unit 12 may generate and display a question sentence for the sentence instructed by the user based on the clinical information of the similar patient. As a result, for example, even in a case where the instruction sentence is not appropriate due to the fluctuation of terms or the like, the input of the appropriate instruction sentence can be received again from the user. In this case, the control unit 12 may cause the AI such as a large language model to learn the clinical information of the similar patient, generate and display to the user a question about fluctuation between the term in the sentence instructed by the user and the term in the medical record included in the clinical information of the similar patient, the classification range of the disease name, and the like.

Subsequently, the control unit 12 outputs information generated from the clinical information of the similar patient based on the sentence instructed by the user (step S105). Here, the control unit 12 may generate content (sentence, graph, image, etc.) of an answer to the sentence instructed by the user based on the clinical information of the similar patient using the large language model (LLM). As a result, for example, the user can know the statistical data of the similar case with an input sense such as a chat.

In this case, the control unit 12 may input the clinical information of the similar patient into the large language model and cause the large language model to learn. Then, the control unit 12 may instruct the large language model to generate an answer to the sentence instructed by the user in an instruction sentence (prompt) based on the clinical information of the similar patient. Then, the control unit 12 may cause the large language model to generate an answer by inputting a sentence instructed by the user to the large language model.

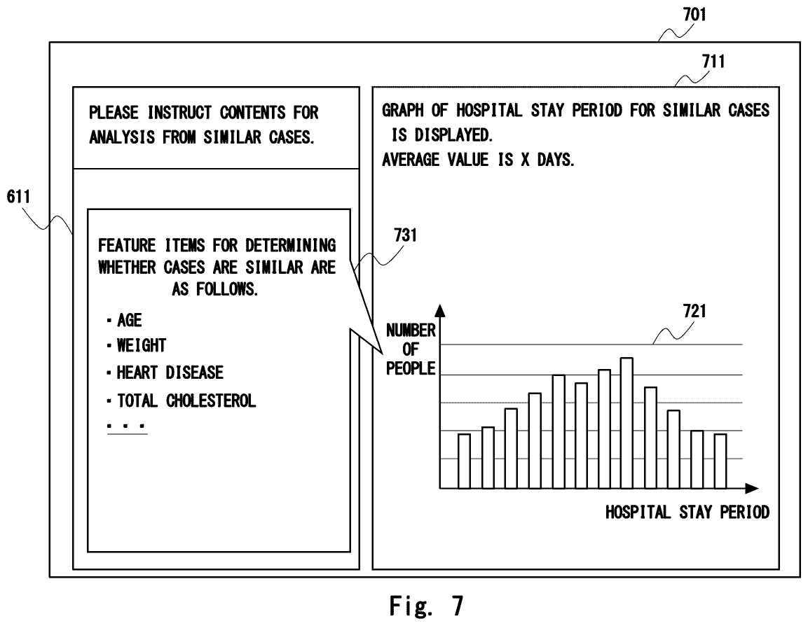

In this case, the control unit 12 may generate information based on the statistical information generated from the clinical information of the similar patient as contents of an answer to the sentence instructed by the user using the large language model. The statistical information may be, for example, a graph showing transition, distribution, or the like of the value of the specific item, or may be a representative value (for example, an average value, a mode value, and a median value) of the specific item value. In the example of FIG. 7, a graph 721 of statistical information is displayed in the display region 711.

The control unit 12 may display information indicating an item used for similarity determination among a plurality of items included in the clinical information of the similar patient in association with the generated information. As a result, for example, the user can appropriately grasp the determination criterion of the similar case that is the original data such as the statistical information. In this case, as illustrated in FIG. 7, the control unit 12 may display a display region 731 of the item used to determine the similarity in association with the graph 721 of the statistical information (In other words, the control unit 12 may cause the display region 731 of the item used to determine the similarity to be displayed in association with the graph 721 of the statistical information).

### Modified Example

The information processing apparatus 10 may be an apparatus included in one housing, but the information processing apparatus 10 of the present disclosure is not limited thereto. Each unit of the information processing apparatus 10 may be achieved by, for example, cloud computing including one or more computers. Such an information processing apparatus 10 is also included in an example of the “information processing apparatus” according to the present disclosure.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each example embodiment can be appropriately combined with other example embodiments.

Some or all of the example embodiments described above may also be described as, but are not limited to, the following Supplementary Notes. Some or all of the elements (for example, configurations and functions) described in each Supplementary Note dependent on Supplementary Note 1 can also be dependent on an independent Supplementary Note of another category in a similar dependency relationship. Some or all of the elements described in any supplementary note may be applied to various types of hardware, software, recording means for recording software, systems, and methods.

### Supplementary Note 1

An information processing apparatus including:
an acquisition unit that acquires clinical information of a plurality of patients; and
a control unit that extracts clinical information of another patient whose similarity to the clinical information of a specific patient is equal to or greater than a threshold, and outputs information generated from the clinical information of the other patient based on a sentence instructed by a user.

### Supplementary Note 2

The information processing apparatus according to Supplementary Note 1, in which the control unit outputs statistical information generated from clinical information of the another patient based on a sentence instructed by the user using a large language model.

### Supplementary Note 3

The information processing apparatus according to Supplementary Note 1 or 2, in which the control unit extracts, based on an item instructed by the user among a plurality of items included in the clinical information of the another patient, clinical information of the another patient whose similarity to the clinical information of the specific patient is equal to or greater than a threshold.

### Supplementary Note 4

The information processing apparatus according to Supplementary Note 1 or 2, in which the control unit displays information indicating an item used for determination of the similarity among the plurality of items included in the clinical information of the another patient in association with the generated information.

### Supplementary Note 5

The information processing apparatus according to Supplementary Note 1 or 2, in which the control unit displays information indicating an item other than the item used for determination of the similarity among the plurality of items included in the clinical information of the another patient in a case where the sentence is instructed by the user.

### Supplementary Note 6

The information processing apparatus according to Supplementary Note 1 or 2, in which the control unit determines and displays a candidate of an instruction sentence relevant to an item designated by the user among the plurality of items included in the clinical information of the another patient.

### Supplementary Note 7

The information processing apparatus according to Supplementary Note 1 or 2, in which the control unit receives setting of an item used for determination of the similarity for each of at least one of a clinical department and a disease name.

### Supplementary Note 8

The information processing apparatus according to Supplementary Note 1 or 2, in which the control unit generates and displays a question sentence for the sentence instructed by the user based on the clinical information of the another patient.

### Supplementary Note 9

An information processing method including:
acquiring clinical information of a plurality of patients; and
extracting clinical information of another patient whose similarity with the clinical information of a specific patient is equal to or greater than a threshold, and outputting information generated from the clinical information of the other patient based on a sentence instructed by a user.

### Supplementary Note 10

A program for causing a computer to execute processing of:
acquiring clinical information of a plurality of patients; and
extracting clinical information of another patient whose similarity with the clinical information of a specific patient is equal to or greater than a threshold, and outputting information generated from the clinical information of the other patient based on a sentence instructed by a user.

While the disclosure has been particularly shown and described with reference to embodiments thereof, the disclosure is not limited to these embodiments. It will be understood by those of ordinary skill in the art that various changes in form and details may be made therein without departing from the spirit and scope of the present disclosure as defined by the claims.

What is claimed is:

1. An information processing apparatus comprising:
at least one storage medium configured to store instructions; and
at least one processor configured to execute the instructions to:
acquire clinical information of a plurality of patients;
extract, based on an item instructed by a user among a plurality of items included in clinical information of another patient, clinical information of the another patient whose similarity to the clinical information of a specific patient is equal to or greater than a threshold; and
output statistical information generated from the clinical information of the another patient based on a sentence instructed by the user using a large language model.

2. The information processing apparatus according to claim 1, wherein the at least one processor is further configured to execute the instructions to:
display information indicating an item used for determination of the similarity among the plurality of items included in the clinical information of the another patient in association with the generated information.

3. The information processing apparatus according to claim 1, wherein the at least one processor is further configured to execute the instructions to:
display information indicating an item other than the item used for determination of the similarity among the plurality of items included in the clinical information of the another patient in a case where the sentence is instructed by the user.

4. The information processing apparatus according to claim 1, wherein the at least one processor is further configured to execute the instructions to:
determine and display a candidate of an instruction sentence relevant to an item designated by the user among the plurality of items included in the clinical information of the another patient.

5. The information processing apparatus according to claim 1, wherein the at least one processor is further configured to execute the instructions to:
receive setting of an item used for determination of the similarity for each of at least one of a clinical department and a disease name.

6. The information processing apparatus according to claim 1, wherein the at least one processor is further configured to execute the instructions to:
generate and display a question sentence for the sentence instructed by the user based on the clinical information of the another patient.

7. An information processing method for causing at least one processor to execute:
acquiring clinical information of a plurality of patients;
extracting, based on an item instructed by a user among a plurality of items included in clinical information of another patient, clinical information of the another patient whose similarity to clinical information of a specific patient is equal to or greater than a threshold; and
outputting statistical information generated from clinical information of the another patient based on a sentence instructed by the user using a large language model.

8. A non-transitory computer-readable medium stored with a program for causing a computer to execute processing of:
acquiring clinical information of a plurality of patients;
extracting, based on an item instructed by a user among a plurality of items included in clinical information of another patient, clinical information of the another patient whose similarity to clinical information of a specific patient is equal to or greater than a threshold; and
outputting information generated from clinical information of the another patient based on a sentence instructed by the user using a large language model.
